(19) United States
(12) Patent Application Publication
(10) Pub. No.: US 2019/0099401 A1
(43) Pub. Date: Apr. 4, 2019

(54) METHOD FOR PROTECTING CORNEAL ENDOTHELIAL CELLS FROM THE IMPACT CAUSED BY AN EYE SURGERY

(71) Applicant: Chang Gung Memorial Hospital, Linkou, Taoyuan City (TW)

(72) Inventors: Hung-Chi CHEN, Taoyuan City (TW); Hui-Kang MA, Taoyuan City (TW); Chao-Min CHENG, Hsinchu (TW); Yi-Jen HSUEH, Taoyuan City (TW)

(21) Appl. No.: 15/920,005

(22) Filed: Mar. 13, 2018

(30) Foreign Application Priority Data
Oct. 3, 2017 (TW) 106134221

(51) Int. Cl. A61K 31/375; A61K 9/00; A61P 27/02; A61P 39/06

(52) U.S. Cl. CPC A61K 31/375; A61P 39/06; A61P 27/02; A61K 9/0048

(57) ABSTRACT

A method for protecting corneal endothelial cells from the impact caused by an eye surgery is disclosed. The ophthalmic composition is administered to a patient's eye continuously for at least five days before an eye surgery for reducing the impact to the corneal endothelial cells caused by the eye surgery. The ophthalmic composition comprises an ascorbic acid and a pharmaceutically acceptable ophthalmic carrier.

### CROSS REFERENCE TO RELATED APPLICATION

This application claims the benefits of the Taiwan Patent Application Serial Number 106134221, filed on Oct. 3, 2017, the subject matter of which is incorporated herein by reference.

### BACKGROUND OF THE INVENTION

### 1. Field of the Invention

The present invention relates to a method for protecting corneal endothelial cells by applying ascorbic acid to a patient's eye, particularly, to a method for protecting corneal endothelial cells from the impact caused by an eye surgery through administering ascorbic acid to a patient's eye.

### 2. Description of Related Art

Cataract is the most common reason causing blurred vision to the elderly person. When the soluble protein in the crystal body becomes insoluble, the crystal body becomes turbid thus causing cataract. According to the statistics from Taiwan's health insurance administration, about 80% of elderlies over 60 years old suffering from cataracts, therefore, the number of executing phacoemulsification is more than any other medical operations, there are about seventeen thousand cases per year. Although technique and the equipment for conducting Phacoemulsification are progressing over time, there are still a number of patients who may experience complications such as corneal edema, ocular hypertension, inflammation, bleeding, etc.

Phacoemulsification is known to cause damages to the corneal endothelial cells, the number of corneal endothelial cells may decrease and their reproducibility in the body is minimal. When the number of corneal endothelial cells is too low, the barrier and pumping function of the endothelium becomes poor; therefore, the eyesight may be damaged due to corneal edema or corneal opacity. Especially for those patients with low corneal endothelial density, the oxidative stress generated from phacoemulsification may cause cell apoptosis or autophagy to the corneal endothelial cells; eventually, the corneal endothelial cells may lose their compensatory effect and cause blindness.

Usually, eye drops comprising steroid are commonly used to prevent or treat the complications of corneal endothelial cells from operations, however, the side effects of steroid cannot be ignored. The artificial vitreous body used during the surgery may reduce the loss of corneal endothelial cells, however, the protective effect only work during the operation. If the artificial vitreous body was left over in the anterior chamber, it may cause more serious complications such as glaucoma.

Accordingly, a method for protecting corneal endothelial cells from the impact caused by the oxidative stress generated from phacoemulsification is needed to increase the success rate of operation and effectively prevent complications.

### SUMMARY OF THE INVENTION

In order to solve the aforementioned problems, the present invention provides a method for protecting corneal endothelial cells, which comprises the step of administering an ophthalmic composition comprising ascorbic acid. The ophthalmic composition is administered continuously to the eye before an eye surgery, such as phacoemulsification, so that the anterior chamber fluid may have sufficient ascorbic acid. Ascorbic acid may inhibit cell apoptosis and autophagy of corneal endothelial cells that are caused by the oxidative stress. Accordingly, the ophthalmic composition may protect the corneal endothelial cells and avoid complications caused by the loss of corneal endothelial cells before and after the eye surgery.

The present invention provides a method of protecting corneal endothelial cells, which comprises the step of administering an ophthalmic composition to a patient's eye continuously for at least five days before an eye surgery to reduce the impact to the corneal endothelial cells caused by the eye surgery, wherein the ophthalmic composition mainly comprises an ascorbic acid and a pharmaceutically acceptable ophthalmic carrier.

In one embodiment, the concentration of the ascorbic acid in the ophthalmic composition is 10 mg/cc to 60 mg/cc, wherein 20 mg/cc to 50 mg/cc is preferable.

The concentration of the ascorbic acid in the ophthalmic composition may be adjusted according to the duration days and the number of times per day of administering the ophthalmic composition. That is, if the concentration of ascorbic acid in the ophthalmic composition is lower, the duration days of administering the ophthalmic composition may be longer and the number of times of administering the ophthalmic composition per day may be more. On the contrary, if the concentration of ascorbic acid in the ophthalmic composition is higher, the duration days of administering the ophthalmic composition may be shorter and the number of times of administering the ophthalmic composition per day may be fewer. The concentration of ascorbic acid in the ophthalmic composition, the duration days of administering the ophthalmic composition, and the number of times of administering the ophthalmic composition per day may be adjusted by skilled professionals in the art. In one preferred embodiment, the ophthalmic composition is administered to the patient's eye continuously for at least 28 days and at least four times a day before the eye surgery for achieving a better protective effect to the corneal endothelial cells.

For example, when the concentration of ascorbic acid in the ophthalmic composition is 50 mg/cc, the ophthalmic composition may be administered to the patient's eye continuously for 28 days and at least four times a day; when the concentration of ascorbic acid in the ophthalmic composition is 20 mg/cc, the ophthalmic composition may be administered to the patient's eye continuously for 28 days and at least 12 times a day. However, the present invention is not limited thereto.

In one embodiment, the eye surgery is phacoemulsification. Phacoemulsification is a small incision surgery using ultrasonic to emulsify the turbid crystal and absorb the turbid crystal after emulsification, an artificial crystal is then implanted into the patient's eye so that the patient may see clearly again. The ophthalmic composition of the present invention may reduce the damages to the corneal endothelial cells caused by oxidative stress generated from the phacoemulsification, for example, cell apoptosis and autophagy of corneal endothelial cells caused by the oxidative stress may be inhibited.

In one embodiment, the pharmaceutically acceptable ophthalmic carrier is selected from a group consisting of aqueous solution, hydrogel, ointment, and liposome.

In addition, the ophthalmic composition of the present invention is selectively comprises buffer solutions, antimicrobial agents, viscosity agents, wetting agents, surfactants, or other additives in the art. Buffer solutions may be added to maintain the pH value of the ophthalmic composition within a suitable range, for example, phosphate buffer, borate buffer, acetate buffer, citrate buffer, or other buffers known in the art may be used. Antimicrobial agents may be added to prevent bacterial growth, for example, penicillins, lincolines, polypeptide antibiotics, tetracyclines, sulfonamides, antivirals, or other anti-microbial agents known in the art may be used. Viscosity agents may be added to adjust the viscosity of the ophthalmic composition to improve the convenience when administering the ophthalmic composition to the eye, for example, sodium carboxymethylcellulose, methylcellulose, hydroxypropylmethylcellulose, polyvinyl alcohol, dextran, polyacylic acid, or other viscosity agents known in the art may be used. Wetting agents may elongate the time for the ophthalmic composition to be present in the eye, for example, sodium carboxymethylcellulose, hydroxypropylmethylcellulose, hydroxyethylcellulose, methylcellulose, polyvinyl alcohol, povidone, glycerol, propylene glycol, or other wetting agents known in the art may be used. Surfactants may be added to reduce the surface tension of the ophthalmic composition so that the composition may completely contact with the eyeball, for example, sodium chloride, potassium chloride, glycerol, mannitol, sorbitol, sodium borate, or other surfactants known in the art may be used.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the result of the cell experiment of an embodiment of the present invention;

FIG. 2 shows the result of another cell experiment of an embodiment of the present invention;

FIG. 3 shows the result of the oxidative stress test of an embodiment of the present invention;

FIG. 4 shows the result of the safety test of an embodiment of the present invention.

### DETAILED DESCRIPTION OF THE PREFERRED EMBODIMENT

[Cell Experiment]

In the present embodiment, retinal pigment epithelial cell line (ARPE19) and corneal endothelial cell line (B4G12) were subjected to the cell experiment. The B4G12 cells were cultured with the cell culture medium comprising Human Endothelial-SFM, 2% of fetal bovine serum (FBS), and 10 ng/ml of basic fibroblast growth factor (bFGF). The culture medium was changed every two days. The ARPE 19 cells were cultured with the cell culture medium comprising Dulbecco's Modified Eagle Medium Nutrient Mixture F-12 (DMEM-F12), 10% of FBS, 0.5% of ampicillin, and 0.125% of gentamycin. The culture medium was changed every two days.

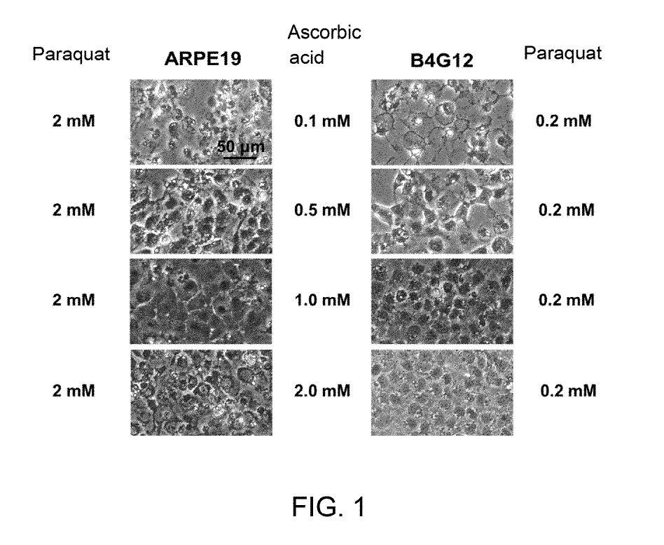

2 mM of paraquat was added to the culture medium of the ARPE19 cells, and 0.2 mM of paraquat was added to the culture medium of the B4G12 cells to induce apoptosis of the cells. 0.1, 0.5, 1.0, and 2.0 mM of ascorbic acid were then added respectively to the culture medium of the cells for 5 days and the cell morphologies thereof were shown in FIG. 1. As the result shown in FIG. 1, the cells added with 0.1 mM or 0.5 mM of ascorbic acid showed cell apoptosis and vesicle formation; however, 1.0 mM and 2.0 mM of ascorbic acid showed the protective effect for the cells. Accordingly, the lowest effective concentration of ascorbic acid is 1.0 mM.

Further, ARPE19 cells and B4G12 cells were divided respectively into four groups, which comprises a control group 1 without added ascorbic acid or paraquat; a control group 2 added with ascorbic acid but without paraquat; a comparative group added with paraquat but without ascorbic acid; and a treatment group added with ascorbic acid and paraquat, wherein 1 mM of ascorbic acid were added to the control groups 2 and treatment groups, 2 mM of paraquat were added to the comparative group and the treatment group of ARPE19 cells, and 0.2 mM of paraquat were added to the comparative group and the treatment group of B4G12 cells.

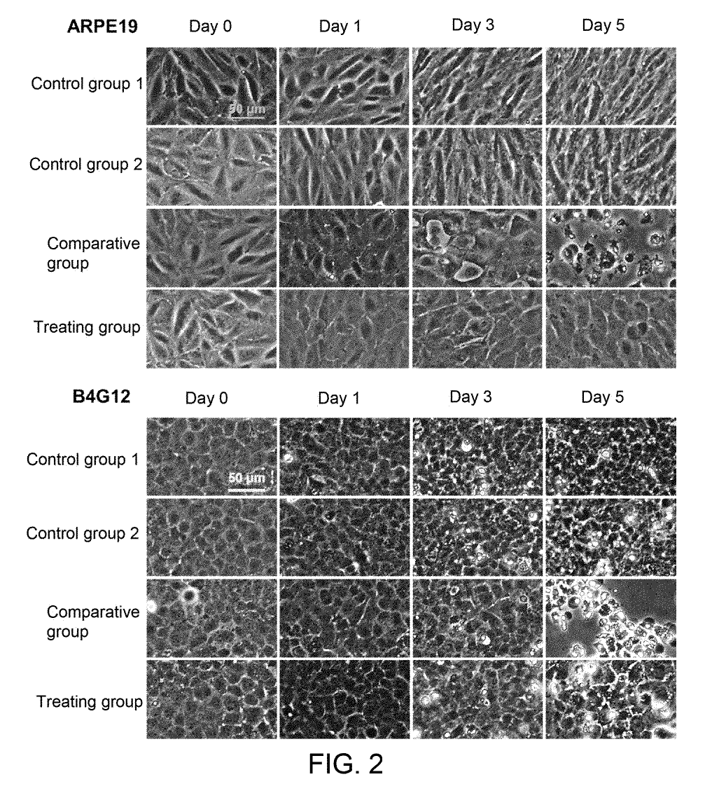

According to the experimental results shown in FIG. 2, the control group 1 and control group 2 of ARPE19 and B4G12 cells without treating paraquat did not show significant cell apoptosis, only some cell vesicles were generated in the control groups 1 and 2 of B4G12 cells. The comparative groups of ARPE19 and B4G12 cells, which were added with 2 mM and 0.2 mM of paraquat respectively, showed significant cell apoptosis; however, cell apoptosis and cell vesicle generation were not significant in the treatment groups of ARPE19 and B4G12 cells, which were added with 1 mM of ascorbic acid. In summary, the results show that ascorbic acid is protective to the cells.

[Oxidative Stress Test]

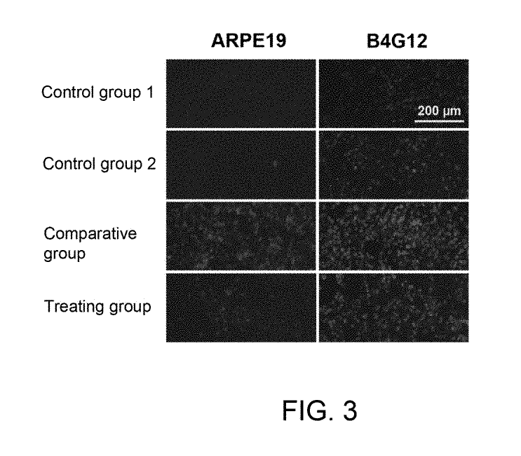

The oxidative stress of ARPE19 and B4G12 cells were tested using cellular ROS detection assay kit (Deep Red Fluorescence, ab186029) after 5 days of culturing. The tested groups were the same as the abovementioned control group 1, control group 2, comparative group, and treatment group. The test results were shown in FIG. 3, wherein the red fluorescent light represented the existence of oxidative stress. According to FIG. 3, the oxidative stress of ARPE 19 of B4G12 cells without adding paraquat did not increase significantly with or without adding ascorbic acid. However, the oxidative stress significantly increased in the groups of ARPE19 cells adding 2 mM of paraquat and B4G12 cells adding 0.2 mM of paraquat, and it also showed that ascorbic acid may sufficiently decrease the oxidative stress caused by paraquat.

[Safety Test]

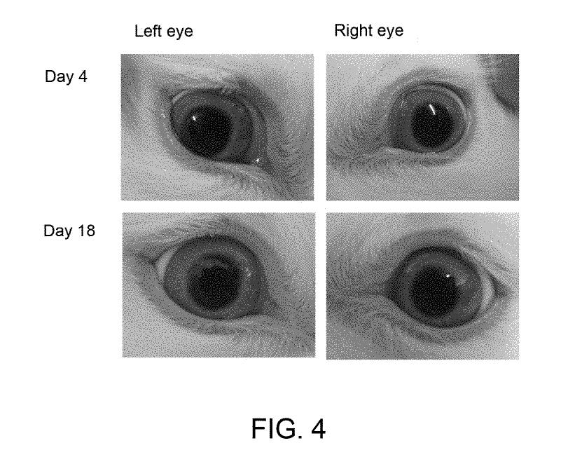

The ophthalmic composition comprising 50 mg/cc of ascorbic acid was administered to both eyes of New Zealand white rabbit once a day continuously for eighteen days. The outer appearance of both eyes of the rabbit was observed on day 4 and day 18 and the results were shown in FIG. 4. According to FIG. 4, there is no obvious trauma in the eyes of the rabbit, which were administered with 50 mg/cc (high concentration) of ascorbic acid. Therefore, the ophthalmic composition of the present invention has no safety concerns when administered to the eyes.

[Phacoemulsification]

Please refer to the following Table. 1 showing the age and sex of the patients, concentration of ascorbic acid in the ophthalmic composition, cause of low corneal endothelial cell density (ECD), total energy of phacoemulsification, duration time of phacoemulsification, corneal endothelial cell density before and after the phacoemulsification, and the percentage of cell loss. The following patients were administered with the ophthalmic composition comprising ascorbic acid continuously for 28 days and four times a day.

TABLE 1

| | Conc. Of | | | | Pre-op/Post- | |
|---|---|---|---|---|---|---|
| | ascorbic | | | Energy/ | op corneal | |
| | acid | | Cause of low | time | ECD | |
| | (mg/cc) | Age/Sex | corneal ECD | (mJ/sec) | (cells/mm²) | Cell lost |
| Patient 1 | — | 45/M | Fuchs dystrophy | 21.3/48.5 | 1481/failed | — |
| OD | | | | | | |
| Patient 2 | 20 | 45/M | Fuchs dystrophy | 24.2/41.4 | 1365/1239 | 9.3% |
| OS | | | | | | |
| Patient 3 | 20 | 54/M | Endotheliitis | 14.5/27.4 | 1039/981 | 5.6% |
| OD | | | | | | |
| Patient 4 | 50 | 73/M | Fuchs dystrophy | 31.6/57.1 | 1406/1355 | 3.6% |
| OD | | | | | | |
| Patient 5 | 50 | 70/F | Fuchs dystrophy | 28.3/35.9 | 1037/983 | 5.3% |
| OD | | | | | | |
| Patient 6 | 50 | 72/M | Endotheliitis | 37.9/59.8 | 1354/1238 | 8.6% |
| OS | | | | | | |
| Patient 7 | 50 | 70/F | Fuchs dystrophy | 29.4/44.6 | 1216/1119 | 8.0% |
| OD | | | | | | |
| Patient 8 | 50 | 79/F | Fuchs dystrophy | 42.5/63.3 | 1351/1249 | 7.6% |
| OS | | | | | | |
| Patient 9 | 50 | 58/F | Fuchs dystrophy | 15.7/38.4 | 1259/1181 | 6.2% |
| OD | | | | | | |
| Patient 10 | 50 | 73/F | Laser iridectomy | 38.9/32.7 | 1824/1724 | 5.5% |
| OD | | | | | | |
| Patient 11 | 50 | 72/M | Endotheliitis | 41.2/37.6 | 1194/1112 | 6.9% |
| OD | | | | | | |
| Patient 12 | 50 | 63/F | Laser iridectomy | 27.3/49.5 | 1738/1649 | 5.1% |
| OD | | | | | | |
| Patient 13 | 50 | 78/M | Fuchs dystrophy | 32.6/54.5 | 887/838 | 5.5% |
| OD | | | | | | |

OD/OS: Right eye/left eye

According to the results shown in Table. 1, patient 1 were not administered the ophthalmic composition comprising ascorbic acid before phacoemulsification, therefore, patient 1 lost a great amount of corneal endothelial cells after phacoemulsification so that the corneal endothelial cell density could not be detected. However, by comparing the corneal endothelial cell density before and after phacoemulsification of patient 2 to patient 13, who were administered with ophthalmic composition comprising ascorbic acid continuously for 28 days, the phacoemulsification caused little amount of cell lost. The ratio of cell loss after may be down regulated to 6.2±1.9%.

In addition, according to Yamazoe et al. (Yamazoe K, Yamaguchi T, Hotta K, Satake Y, Konomi K, Den S, Shimazaki J (2011) Outcomes of cataract surgery in eyes with a low corneal endothelial cell density. J Cataract Refract Surg Dec 37(12):2130-6), for the patients with low corneal endothelial cell density, the average ratio of cell loss after phacoemulsification is 11.5±23.4%. Comparing to the present invention, the average ratio of corneal endothelial cell loss of the patients administered continuously with the ophthalmic composition comprising ascorbic acid is significantly down-regulated to 6.2±1.9%; therefore, the ophthalmic composition of the present invention has an outstanding protective effect to the corneal endothelial cells.

Based on the aforementioned results, the ophthalmic composition comprising ascorbic acid of the present invention is able to protect the corneal endothelial cells, prevent the cell apoptosis caused by the oxidative stress generated by the eye surgery, and reduce the incidence of complications caused by the eye surgery, such as the corneal endothelial cell loss or corneal edema.

What is claimed is:

1. A method for protecting corneal endothelial cells from the impact caused by an eye surgery, which comprising the step of administering an ophthalmic composition to a patient's eye continuously for at least five days before the eye surgery, wherein the ophthalmic composition comprises an ascorbic acid and a pharmaceutically acceptable ophthalmic carrier.

2. The method as claimed in claim 1, wherein the concentration of the ascorbic acid in the ophthalmic composition is 10 mg/cc to 60 mg/cc.

3. The method as claimed in claim 2, wherein the concentration of the ascorbic acid in the ophthalmic composition is 20 mg/cc to 50 mg/cc.

4. The method as claimed in claim 1, wherein the ophthalmic composition is applied to the patient's eye continuously for at least 28 days and at least four times a day before the eye surgery.

5. The method as claimed in claim 1, wherein the eye surgery is Phacoemulsification.

6. The method as claimed in claim 1, wherein the ophthalmic composition reduces the damages to the corneal endothelial cells caused by oxidative stress generated from the eye surgery.

7. The method as claimed in claim 6, wherein the ophthalmic composition inhibits cell apoptosis and autophagy of corneal endothelial cells caused by the oxidative stress.

8. The method as claimed in claim 1, wherein the pharmaceutically acceptable ophthalmic carrier is selected from a group consisting of aqueous solution, hydrogel, ointment, and liposome.
